(19) United States
(12) Patent Application Publication
(10) Pub. No.: US 2011/0130446 A1
(43) Pub. Date: Jun. 2, 2011

(54) INJECTABLE TAXANE PHARMACEUTICAL COMPOSITION

(75) Inventors: Antonio Parente Duena, Barcelona (ES); Josep Garces Garces, Barcelona (ES); Ricard Mis Vizcaino, Barcelona (ES)

(73) Assignee: GP PHARM, S.A., Sant Quinti de Mediona, Barcelona (ES)

(21) Appl. No.: 13/057,661

(22) PCT Filed: Aug. 6, 2009
(86) PCT No.: PCT/EP09/05691

§ 371 Date: Feb. 4, 2011

(30) Foreign Application Priority Data
Aug. 7, 2008 (ES) P200802389

(51) Int. Cl. A61K 31/337; A61K 47/14; A61P 35/04; A61J 1/06

(52) U.S. Cl. 514/449; 514/785; 604/403

(57) ABSTRACT

New injectable taxane pharmaceutical composition. Stable composition with low percentage of ethanol and a lower ethanol/taxane ratio than that of the state-of-the-art taxane pharmaceutical compositions.

### FIELD OF THE INVENTION

This invention is in general within the field of biomedicine and in particular refers to a new injectable taxane pharmaceutical composition and its use for the treatment of cancerous tumors.

### BACKGROUND OF THE INVENTION

It has long been known that taxanes such as paclitaxel and docetaxel have a useful biological effect in the treatment of cancerous tumors. Equally known is the low solubility of these compounds in solvents suitable as injectable, which has led to the development of various formulations containing different surfactants and/or other excipients.

Thus, for example, document EP 0593601 B1 describes a taxane composition containing a surfactant selected from the group formed by polysorbates (Tween®), polyethoxylated castor oils (Cremophor®) and polyoxyethylene glycol esters (Emulphor®) and less than 5% ethanol. Document EP 0593656 B1 describes a taxane composition containing polysorbate and ethanol. Due to stability problems, the commercial composition of docetaxel of these inventions is presented in two vials, one containing a solution of docetaxel in polysorbate 80 and a second vial containing an aqueous ethanol solution. The two vials must be mixed to obtain a premix solution which has a physical and chemical stability of eight hours and must be diluted after mixing in a perfusion bag to be administered afterward (Product characteristics from the docetaxel data sheets). This system therefore involves two dilutions of docetaxel prior to administration, the impossibility of obtaining a ready-to-use composition that can be stored, and difficult handling of a cytotoxic compound, which represents a health risk to the healthcare provider.

Document EP 0671912 B1 describes a double compartment injectable composition for the preparation of a perfusion solution composed in the first compartment of a solution with less than 5% ethanol, a taxane, and a surfactant chosen from polysorbates, ether esters of ethylene oxide, and fatty acids glycerides, and in the second compartment of a solvent chosen among organic solvents with molecular weight below 200 or inorganic salts. According to the examples in this paper, the solution of the first compartment is prepared according to patent EP 0593601 B1. As described in EP 0593601 B1, the solutions of paclitaxel or docetaxel without ethanol have a physical stability ranging between 8 and 100 hours and several months. However, there is no single datum in EP 0593601 B1 or EP 0671912 which mentions a physical and chemical stability of several months or even of one week of an injectable or perfusion composition comprising a taxane, ethanol and a surfactant in one container. Furthermore, as indicated above, the data sheet of docetaxel filed with the European Medicines Agency indicates that the stability of an aqueous solution of docetaxel in ethanol and polysorbate 80 is only eight hours when stored between 2° C. and 8° C. or at room temperature.

Furthermore, document WO 2007/020085 A2 describes compositions for injection or perfusion comprising, in one compartment, a taxane, a physiologically acceptable solvent and a physiologically acceptable surfactant. Optionally, these compositions also contain citric acid. Thus, the vial does not need to be reconstituted and is injected directly into the perfusion bag. This document indicates that the composition degrades less than 5% when stored for three months at 40° C. According to a preferred embodiment of this document, the composition comprising a taxane, ethanol and a surfactant other than polysorbate would have a minimum percentage of ethanol of at least 18% by weight and at least an ethanol/taxane weight ratio of 7.9/1. On the other hand, the amount of surfactant in the composition is 12 to 35 times the amount of taxane by weight, although this ratio depends on the surfactant, and in the particular cases of Solutol® HS15 and Tween® 80 the amounts of surfactant in the examples are 30 and 26 times the amount of taxane by weight, respectively. Similarly, the percentages of ethanol in the compositions of the examples in this document vary between 34% and 58% by weight, with ethanol/taxane weight ratios varying between 16.5/1 and 26.3/1. If the treatment requires the administration of high doses of taxanes, it would require the administration of a large amount of the composition and therefore of ethanol.

Document WO 00/20036 A1 describes a pharmaceutical composition comprising paclitaxel, water, an acid, preferably citric acid, and one or more organic solvents, preferably triacetin, glycerin, ethanol and Solutol® HS15. This document extrapolates from the degradation values at 70° C. for 16-24 hours that the stability of a composition according to the invention at room temperature will be greater than 18 months. Likewise, the amount of Solutol® HS15 in the composition, according to the detailed description of this document, can be concluded to be between 50 and 200 times the amount of paclitaxel by weight. Moreover, the amount of ethanol absolute varies between 12.5 and 166.7 times the amount of paclitaxel by weight (Table 1). Thus, administration of high doses of paclitaxel would involve the administration of high quantities of ethanol.

Furthermore, document EP 0876145 A1 describes a solution of taxanes that can be stored and which includes a taxane, ethanol, a polyoxyethylene sorbitan fatty acid monoester (Tween®) and polyethoxylated castor oil (Cremophor®). According to a preferred embodiment, the solution of the invention contains between 15% to 30% ethanol by volume, and it can be calculated that the amount of ethanol is between 19.7 and 59 times the amount of taxane. According to the formulations of the examples in this document, the ethanol/paclitaxel ratio is 26.3/1 by weight.

Document WO 2005/020962 A1 describes compositions administered via any route, comprising a water-insoluble therapeutic agent, particularly a taxane, vitamin E, ethanol, a bioavailability enhancer and tyloxapol or mixtures of tyloxapol and TPGS (vitamin E succinate esterified with polyethylene glycol) as surfactant. According to an embodiment in the description, the amount of paclitaxel is between 0.5 and 4% of the composition by weight, preferably between 1.5% and 3% by weight. According to another embodiment in the description of the invention, the relative proportion of ethanol is between 5% and 50% of the final composition by weight, preferably equal to 30% by weight, although this embodiment does not mention which therapeutic agents could be dissolved in only 5% ethanol or in what amounts. According to the examples in this document in which ethanol and taxanes appear together, it can be calculated that the amount of ethanol is 10 to 20 times the amount of paclitaxel by weight and represents 30% of the composition by weight of the examples.

Furthermore, document WO 2005/097105 A1 describes an injectable taxane composition comprising polyethylene glycol 15-hydroxystearate (Solutol® HS15) and glycofurol as solubilizers, and this composition improves solubility while minimizing toxicity. Additionally, this composition contains an acid agent to adjust the pH of the solution, preferably between 4 and 6. However, the stability of the compositions of the examples containing paclitaxel is not pharmaceutically acceptable, since they present a degradation which in the best case is higher than 0.7% after only 48 hours at 40° C.

Documents DE 19925211 A1, EP 0835657 A1, EP 0674510 A1, WO 03/053350 A2, WO 03/022247 A1, WO 2005/020962 A1 relate to taxane solutions with different solvents and surfactants containing acids to improve the chemical stability of the solutions.

On the other hand, some surfactants can present problems of toxicity, hypersensitivity or dyspnea. Therefore, there is still a need to find a physically and chemically stable taxane pharmaceutical composition that allows a reduction in the levels of ethanol and surfactant of the state-of-the-art pharmaceutical compositions, while reducing the number of manipulations to be performed in order to prepare the final injectable solution.

Documents U.S. 2004/127551 A1, WO 01/72299 A1, WO 03/057208 A1, EP 1479382 A1 and WO 00/78247 A1 describe orally administered compositions comprising a taxane and a vehicle, preferably a surfactant, and a co-solubilizer. In particular, the compositions of the first three documents also contain a stabilizer. The co-solubilizers of the compositions of the five documents are agents that reduce the viscosity of the composition and increase the fluidity of the composition at the temperature of the human body. The co-solubilizers reducing viscosity include surfactants, ethanol, water and citrate esters such as tributyl citrate, triethyl citrate and acetyl triethyl citrate, among others. The amount of co-solubilizer in the compositions can be up to 90% of the composition by weight. In a preferred embodiment, the co-solubilizer is ethanol and the composition comprises 5 to 50% ethanol by weight, preferably 10 to 30% by weight and more preferably 20% by weight. But according to the preferred embodiments and the embodiments of the examples, it is noted that the compositions contain from 19.5 to 22.4% ethanol by weight and the amount of ethanol is between 16.2 and 18.6 times the amount of paclitaxel. It is also necessary to mention here the problems of absorption and tolerance affecting formulations of orally administered paclitaxel up to now, which have so far prevented the marketing of an orally administered paclitaxel drug.

Document EP 1194120 A1 describes a pharmaceutical composition comprising a triglyceride, a carrier comprising at least two surfactants, one of them hydrophilic, and a therapeutic agent including paclitaxel among other. Optionally, this composition may include a solubilizer that increases the solubility of the therapeutic agent or of the triglyceride of the composition. Among the mentioned solubilizers are triethyl citrate, tributyl citrate, acetyl triethyl citrate and acetyl tributyl citrate, although it is not shown either in the description or in the examples in that document that those alkyl citrate esters are solubilizers of taxanes in pharmaceutical compositions including ethanol.

Surprisingly we found that the addition of alkyl esters of citric acid allows obtaining taxane pharmaceutical compositions which are stable and suitable for parenteral administration, with low ethanol content and a lower concentration of surfactants.

### DESCRIPTION OF THE INVENTION

This invention provides stable pharmaceutical compositions for parenteral administration of taxanes, which are useful for the treatment of cancerous tumors.

Thus, a first aspect of this invention refers to a stable pharmaceutical composition for parenteral administration, comprising a therapeutically effective amount of taxane, at least one surfactant, at least one alkyl ester of citric acid and ethanol

In the present invention “taxane” means paclitaxel, docetaxel, their derivatives, analogues, metabolites, prodrugs, hydrates and their salts. In one particular embodiment, the taxane in the composition of the invention is selected from the group consisting of paclitaxel, docetaxel anhydrous or docetaxel trihydrate, preferably docetaxel anhydrous or docetaxel trihydrate.

In this invention “stable” means that the composition meets the pharmaceutical stability criteria set out in version Q1A(R2) of Feb. 6, 2003 by the International Conference on Harmonization of Technical Requirements for Registration of Pharmaceuticals for Human Use.

In one particular embodiment, the amount of ethanol in the composition of the invention is less than 20% of the composition by weight and preferably less than 15% of the composition by weight.

In one particular embodiment, the concentration of taxane in the composition of the invention is between 0.05 mg/ml and 220 mg/ml, preferably between 10 mg/ml and 80 mg/ml. In a preferred embodiment, the amount of taxane in the composition of the invention is between 2% and 5% of the composition by weight.

In another particular embodiment, the amount of ethanol in the composition by weight of the invention is less than seven times the amount of taxane by weight, preferably less than five times the amount of taxane by weight and more preferably less than three times the amount of taxane by weight.

In another particular embodiment, the surfactant in the composition of the invention is any non-ionic surfactant that is pharmaceutically acceptable and known in the state of the art, including, for example and not limited to monoesters of polyoxyethylene sorbitan fatty acids (Tween®, Emalex, Nikkol, Hodag, Dacol or Liposorb), monoesters of sorbitan fatty acids (Span®), polyethylene glycol 15-hydroxystearate (Solutol® HS15), polyethylene glycol esters of fatty acids (Crodet, Cithrol, Kessco®, Nikkol, Mapeg®, Myrj, Tagat®, Aldo®, Capmul®, Glycerox, Lactomul® or Emerest®), polyoxyethylene glycol esters (Emulphor®), polyethoxylated castor oils (Cremophor®, Emalex, Eumulgin®, Nikkol, Cerex or Simusol®), polyglycerol esters of fatty acids (Nikkol Decaglyn, Polymuls, Caprol®), polyethylene glycol ethers (Volpe or Brij®), poloxamers (Lutrol® or Pluronic®), polyoxyethylene phenyl ethers (Triton® or Igepal®), or mixtures thereof. Preferably the surfactant is selected from the group consisting of Tween®, Solutol® HS15, Lutrol®, Cremophor® or mixtures thereof, more preferably from the group consisting of Tween® and Solutol® HS15.

The total amount of surfactants in the composition of the invention depends on the hydrophilic/lipophilic balance of each surfactant and its molecular weight. In one particular embodiment, the total amount of surfactants in the composition is 1 to 50 times the amount of taxane in the composition by weight, preferably 10 to 30 times the amount of taxane by weight, and in particular 15 to 20 times the amount of taxane by weight. In one particular embodiment, when the surfactant in the composition of the invention is only Solutol® HS15, the amount of surfactant is 10 to 25 times the amount of taxane in the composition by weight, preferably 15 to 20 times by weight. In one particular embodiment, when the surfactant in the composition of the invention is only Tween® 80, the amount of surfactant is 7 to 15 times the amount of taxane in the composition by weight, preferably 10 to 12 times by weight.

In this invention, the term “alkyl ester of citric acid” refers to a citrate, the citrate being substituted or not by an acetyl group, where the alkyl part of the ester is a saturated linear or branched hydrocarbon group, including, for example and not limited to triethyl citrate, acetyl triethyl citrate, tributyl citrate, acetyl tributyl citrate, trimethyl citrate, trihexyl citrate, acetyl trihexyl citrate, trioctyl citrate, acetyl trioctyl citrate or mixtures thereof. Preferably the alkyl ester of citric acid is selected from the group consisting of triethyl citrate, acetyl triethyl citrate, tributyl citrate.

In another particular embodiment, the total amount of alkyl esters of citric acid is 0.1 to 10 times the amount of taxane in the composition by weight, preferably 1 to 5 times the amount of taxane in the composition by weight.

In another particular embodiment, the amount of ethanol in the composition of the invention is between 5% and 15% of the composition by weight, preferably between 7% and 12% by weight.

In another particular embodiment, the pharmaceutical composition of the invention additionally comprises at least one acid to lower the pH of the solution to a value between 2 and 6, preferably between 2.5 and 4.5. The acid optionally comprised in the composition of the invention is any mineral acid, organic acid and/or amino acid that allows obtaining the abovementioned pH, such as for example, and not limited to hydrochloric acid, nitric acid, phosphoric acid, sulfuric acid, benzoic acid, benzene sulfonic acid, citric acid, ascorbic acid, diatrizoic acid, tartaric acid, lactic acid, hydracrylic acid, formic acid, maleic acid, succinic acid, oxalic acid, malic acid, malonic acid, mandelic acid, pyruvic acid, aspartic acid, glutamic acid, glycine, histidine, tyrosine, or mixtures thereof. In a preferred embodiment, the acid added to the composition of the invention is selected from the group consisting of hydrochloric acid, citric acid, lactic acid, aspartic acid and glycine.

In another particular embodiment, the parenteral administration of the composition of the invention is performed by bolus, infusion and/or intravenous perfusion, with preferred administration via solution for perfusion.

In another particular embodiment, the composition of the invention is presented as an homogeneous solution in the form of a pre-filled syringe, vial, ampoule, bottle, perfusion bag or other packaging material known to the man of the art.

The composition of this invention can be prepared by any method known in the state of the art. In particular, the composition of this invention is prepared under sterile conditions.

In another aspect, the pharmaceutical composition of this invention is used for the treatment of any type of cancerous tumor for which taxanes are effective in the inhibition of the growth and/or elimination of the tumor, for example, and not limited to, breast carcinoma, ovarian carcinoma, bladder carcinoma, kidney carcinoma, prostate carcinoma, stomach carcinoma, colon carcinoma, pancreatic carcinoma, liver carcinoma, lung carcinoma, Kaposi's sarcoma, melanoma, carcinoma of the neck, throat and mouth, brain carcinoma, glioblastoma and lymphoma.

According to another aspect, this invention relates to the use of the composition of the invention in the preparation of a medicinal product for the treatment of any type of cancerous tumor for which taxanes are effective in the inhibition of the growth and/or elimination of the tumor, for example, and not limited to, breast carcinoma, ovarian carcinoma, bladder carcinoma, kidney carcinoma, prostate carcinoma, stomach carcinoma, colon carcinoma, pancreatic carcinoma, liver carcinoma, lung carcinoma, Kaposi's sarcoma, melanoma, carcinoma of the neck, throat and mouth, brain carcinoma, glioblastoma and lymphoma.

According to another aspect, this invention relates to a method for treating cancerous tumors which comprises administering a therapeutically effective amount of the pharmaceutical composition of the invention in adjuvant therapy or in combination with one or more drugs which contain a therapeutically effective amount of an agent for the treatment of cancer. Among the agents for the treatment of cancer are included, for example, and not limited to, doxorubicin, cyclophosphamide, trastuzumab, capecitabine, cisplatin, prednisone, prednisolone, 5-fluorouracil, leuprolide, buserelin, goserelin, histrelin or triptorelin.

In another aspect of the invention, we found that the direct dilution of the composition of the invention in an aqueous carrier produces a solution for perfusion characterized in that it is a micro-emulsion or micro-suspension containing at least one surfactant, a taxane, at least one alkyl ester of citric acid, ethanol and optionally an acid. In a preferred embodiment, the aqueous carrier is selected from the group consisting of 5% dextrose serum and 0.9% saline serum. In another preferred embodiment, the maximum of the distribution curve of particle size in the micro-emulsion or micro-suspension is between 0.2 nm and 40 nm, preferably between 2 nm and 30 nm, and more preferably between 5 nm and 15 nm.

The dose of the composition of the invention to be administered depends on several factors, including the taxane in particular, patient status, patient weight, the severity of the tumor to be treated, the form and frequency of administration. In a preferred embodiment, the concentration of docetaxel in the composition for perfusion is between 0.05 mg/ml and 1.2 mg/ml, preferably between 0.05 mg/ml and 0.74 mg/ml, administering up to 100 mg/m² every three weeks. In another preferred embodiment, the concentration of paclitaxel in the composition for perfusion is between 0.05 mg/ml and 1.2 mg/ml, administering up to 220 mg/m² every three weeks.

### DETAILED DESCRIPTION OF THE INVENTION

Surprisingly, we have found a stable pharmaceutical composition for parenteral administration, comprising a therapeutically effective amount of a taxane, at least one surfactant, at least one alkyl ester of citric acid and ethanol in an amount less than 15% by weight.

### EXAMPLES

These examples are intended to be illustrative of the invention and not limited thereto

### Example 1

### Pharmaceutical Composition of Docetaxel Prepared from a First Procedure

20 g of docetaxel anhydrous were introduced in a 10-L capacity reactor equipped with mechanical stirring and heating jacket pre-heated to 30° C. 75 g of tributyl citrate, 26 g of ethanol absolute, 4 g of aspartic acid and 240 g of Tween® 80 previously melted by heating to 40° C. were added about it. The mixture was vigorously stirred until all the docetaxel anhydrous was dissolved completely. The resulting solution was sterilized by filtration through a 0.22 μm pore diameter absolute filter, and dosed.

### Example 2

### Pharmaceutical Composition of Docetaxel Prepared from a Second Procedure

350 g of Lutrol® F 68 were placed in a stainless steel or glass container equipped with a tight lid, mechanical stirring and heating jacket and the contents were heated to 120° C. for 30 minutes to ensure sterilization of the product. The reactor contents were subsequently cooled to reach a temperature between 30 and 35° C.

20 g of docetaxel anhydrous, 75 g of acetyl triethyl citrate, 7 g of lactic acid and 70 g of ethanol absolute were placed in another hermetically sealed container equipped with mechanical stirring and heating jacket. The mixture was stirred until all the docetaxel was completely dissolved. The resulting solution was transferred through a 0.22 μm pore diameter sterilizing filter inside the container with sterile Lutrol® F 68.

The contents of the reactor were stirred for 30 minutes until a homogeneous and totally transparent solution was obtained, and dosed.

### Example 3

### Pharmaceutical Composition of Docetaxel Prepared from a Third Procedure

350 g of Solutol® HS15 were placed in a stainless steel or glass container equipped with tight lid, mechanical stirring and heating jacket, and the contents were heated to 30-35° C. until completely melted.

20 g of docetaxel trihydrate, 85 g of trihexyl citrate and 65 g of ethanol absolute were placed in another hermetically sealed container equipped with mechanical stirring and heating jacket. The mixture was stirred until all the docetaxel was completely dissolved. Both solutions were mixed and stirred gently until total homogenization.

The resulting solution was sterilized by filtration through a 0.22 μm pore diameter sterilizing filter, and dosed.

### Example 4

### Pharmaceutical Composition of Docetaxel

The composition was prepared by the same procedure as Example 1, but starting from 20 g of docetaxel anhydrous, 60 g of triethyl citrate, 50 g of ethanol absolute, 6 g of citric acid and 350 g of Solutol® HS15.

### Example 5

### Pharmaceutical Composition of Paclitaxel

The composition was prepared by the same procedure as Example 1, but starting from 20 g of paclitaxel, 75 g of triethyl citrate, 26 g of ethanol absolute and 200 g of Cremophor® EL.

### Example 6

### Stability Example

The pharmaceutical composition prepared according to example 4 was subjected to storage conditions at 5° C. and accelerated aging at 25° C. as described in document Q1A (R2) for stability testing for new pharmaceutical drugs from the International Conference on Harmonization of Technical Requirements for Registration of Pharmaceuticals for Human Use.

The stability analysis was performed by HPLC using the following chromatographic method:
Eluents: Water and acetonitrile,
Column: Waters symmetry C18 3.5 μm 4.6×100 mm
Temperature: 30° C.
Flow: 1.0 mL/min.
Detection: UV to 225 nm
Isocratic: A/B 60:40

The results are summarized in table 1:

TABLE 1

| Study | | Total impurities after six |
|---|---|---|
| temperature | Initial total impurities | months |
| 5° C. | 0.72% | 0.7% |
| 25° C. | 0.72% | 0.89% |

### Example 7

### Preparation of a Solution for Perfusion

For the administration of the product in humans, it is necessary to perform a previous dilution of the preparation in a solution of 5% glucose or in a solution of 0.9% sodium chloride.

This is done by taking a perfusion bag or bottle containing 250 ml of the solution of 5% glucose or the solution of 0.9% sodium chloride. An appropriate volume of any of the solutions obtained in Examples 1 to 5 is injected into this perfusion bag or bottle. The perfusion bag or bottle must then be subjected to gentle stirring until a totally transparent solution is obtained. The volume of preparation injected into the bag or bottle for perfusion must be enough for the drug concentration in the perfusion bag to be less than 1.2 mg/ml of taxane. If administration requires a dose greater than 250 mg of active substance, then it is necessary to prepare a higher volume of solution for perfusion.

### Example 8

### Measurement of Average Particle Size

The particle size in the composition for perfusion of Example 6 in 0.9% saline serum for the composition of example 4 was measured on Zetasizer Nano S equipment using Dynamic Light Scattering (DLS) technique. Table 2 shows the values of particle size at different times according to the Z average parameter.

TABLE 2

| Time (hours) | Z average (nm) |
|---|---|
| 0 | 11.7 |
| 1 | 11.6 |
| 2 | 11.6 |
| 4 | 11.9 |

### Example 9

### Comparative Toxicity Study

A comparative toxicity study was performed in rats to which either the composition of example 4 or the formulation of Taxotere® was administered in single doses 2.75 times the maximum human dose. We evaluated the mortality, clinical signs, weight changes, haematology and macroscopic pathology for 14 days, with similar results for both formulations.

### Example 10

### Pharmaceutical Vehicle Composition

A composition was prepared only with excipients and adjuvants of the composition of Example 4. The composition was prepared as the composition of example 4, but starting from 60 g of triethyl citrate, 50 g of ethanol absolute, 6 g of citric acid and 350 g of Solutol® HS15.

### Example 11

### Taxotere® Vehicle Composition

A composition was prepared only with the excipients and adjuvants of the Taxotere® composition (approved docetaxel composition by the European Medicines Agency and FDA) starting from 19.08 g of ethanol absolute, 54 g of polysorbate 80 and 127.37 g of water.

### Example 12

### Comparative Toxicity Case

The aim of this study was to obtain information about the cumulative toxicity of test items over a period of 28 days. A 28 day-intravenous toxicity study in Sprague Dawley Rat, daily administration of a dose (2.5 times maximum human dose) of the composition of example 10 versus the composition of example 11 was done. Additionally, this study provided a rational base for evaluation of the toxicological risk in humans, also indicating potential target organs.

The maximum human dose of docetaxel is 2.7 mg/kg, the equivalent doses in rat to the human dose was calculated using the body surface area-based conversion (factor of 6.2). The amount of administrated docetaxel in Sprague Dawley Rat would have been 41.85 mg/kg. The administrated amounts of the excipients and adjuvants of the composition of example 10 and the excipients and adjuvants of the composition of example 11, but keeping the ratio with 41.85 mg/kg of docetaxel, are shown in table 3.

TABLE 3

| Composition of example 10 | | Composition of example 11 | |
|---|---|---|---|
| Triethyl citrate: | 125.55 mg/kg | Ethanol absolute: | 399.28 mg/kg |
| Ethanol absolute: | 104.625 mg/kg | Polysorbate 80: | 1130.26 mg/kg |
| Citric acid: | 12.555 mg/kg | Water: | 2665.38 mg/kg |
| Solutol ® HS15: | 732.375 mg/kg | | |

### Results:

See table 4

TABLE 4

| | Composition of example 10 | Composition of example 11 |
|---|---|---|
| | (9 males + 10 females) | (10 males + 10 females) |
| Clinical | 2 males and 1 female were | 1 male and 2 females were found |
| signs | found death on day 25 | death on days 17, 2 and 13, |
| | | respectively. |
| | | Wound at the site of injection in |
| | | the tail of 2 males and 1 female |
| | | (day 12). |
| Haema- | | Significant decrease in white |
| tology | | blood cell concentration in |
| | | females. |

### Example 13

### Comparative Subacute Toxicological Study in Beagle Dogs

A comparative subacute toxicological study between the composition of example 4 and Taxotere® in beagle dogs was done in a five-day repeated-dose administration. The purpose of this study was to compare the subacute toxicity of the composition of example 4 against the reference item Taxotere® when intravenously administered once a day for 5 consecutive days.

A summary of the study plan is described in table 5.

TABLE 5

| Animal model | |
|---|---|
| Groups (3 males + 3 females/group) | Docetaxel concentration |
| 1. Low dose composition of example 4 | 3 mg/m² (0.15 mg/kg) |
| 2. High dose composition of example 4 | 6 mg/m² (0.30 mg/kg) |
| 3. Low dose Taxotere ® | 3 mg/m² (0.15 mg/kg) |
| 4. High dose Taxotere ® | 6 mg/m² (0.30 mg/kg) |

### Results:

1. Mortality: No mortality was recorded.
2. Clinical signs: See table 6

TABLE 6

| | | Clinical signs (number of |
|---|---|---|
| | | animals affected) |
| Composition | Group 1 | Salivation (2) |
| Example 4 | (0.15 mg/kg) | Lacrimation (2) |
| | Group 2 | Mucous faeces (2) |
| | (0.30 mg/kg) | Salivation (1) |
| | | Lacrimation (1) |
| Taxotere ® | Group 3 | Decreased motor activity (3) |
| | (0.15 mg/kg) | Head shaking (6) |
| | | Pruritus (3) |
| | | Skin red (6) |
| | | Reddish (right and left eye mucous |
| | | membrane) (2) |
| | | Salivation (5) |
| | | Lacrimation (3) |
| | | Pasty faeces (3) |
| | | Mucous faeces (1) |
| | | Pain during administration (2) |
| | | Tremor (1) |
| | | Vomiting (1) |
| | Group 4* | Decreased motor activity (3) |
| | (0.30 mg/kg) | Head shaking (6) |
| | | Pruritus (5) |
| | | Skin red (6) |
| | | Salivation (3) |
| | | Lacrimation (2) |
| | | Pasty faeces (4) |
| | | Pain during administration (3) |
| | | Tremor (1) |
| | | Vomiting (2) |

*Clinical signs in group 4 were more exacerbated.

### Example 14

### Comparative Subacute Toxicological Study in Mice

A comparative subacute toxicological study between the composition of example 4 and Taxotere® in mice was done in a five-day repeated-dose administration. The purpose of this study was to compare the subacute toxicity of the composition of example 4 against the reference item Taxotere® when intravenously administered daily for 5 consecutive days.

A summary of the study plan is described in table 7.

TABLE 7

| Groups (10 males + 10 females/group) | Docetaxel concentration |
|---|---|
| 1. Composition of example 4 | 30 mg/m² (10 mg/kg) |
| 2. Taxotere ® | 30 mg/m² (10 mg/kg) |

### Results:

1. Mortality: No mortality was recorded.
2. Clinical signs: See table 8

TABLE 8

| | Clinical signs (number of |
|---|---|
| | animals affected) |
| Composition of Example 4 | Hunched back (5) |
| Group 1 (10 mg/kg) | Ruffled fur (2) |
| Taxotere ® Group 2 (10 | Hunched back (9) |
| mg/kg) | Ruffled fur (5) |
| | Abnormal gait (2) |

1. Stable pharmaceutical composition for parenteral administration, comprising a taxane, at least one surfactant, at least one alkyl ester of citric acid, and ethanol.

2. Pharmaceutical composition according to claim 1, wherein the amount of ethanol is less than 20% by weight.

3. (canceled)

4. Pharmaceutical composition according to claim 1, wherein the taxane is selected from the group consisting of paclitaxel, docetaxel anhydrous or docetaxel trihydrate.

5. Pharmaceutical composition according to claim 1, wherein the concentration of taxane is between 0.05 mg/ml and 220 mg/ml.

6. (canceled)

7. Pharmaceutical composition according to claim 1, wherein the amount of ethanol by weight is less than seven times the amount of taxane by weight.

8.-9. (canceled)

10. Pharmaceutical composition according to claim 1, wherein the surfactant is selected from the group consisting of monoesters of polyoxyethylene sorbitan fatty acid, monoesters of sorbitan fatty acids, polyethylene glycol 15-hydroxystearate, polyethylene glycol esters of fatty acids, polyoxyethylene glycol esters, polyethoxylated castor oils, polyglycerol esters of fatty acids, polyethylene glycol ethers, poloxamers, polyoxyethylene phenyl ethers or mixtures thereof.

11. Pharmaceutical composition according to claim 1, wherein the total amount of surfactants is 1 to 50 times the amount of taxane by weight.

12-13. (canceled)

14. Pharmaceutical composition according to claim 1, wherein the alkyl ester of citric acid is selected from the group consisting of triethyl citrate, acetyl triethyl citrate, tributyl citrate, acetyl tributyl citrate, trimethyl citrate, trihexyl citrate, acetyl trihexyl citrate, trioctyl citrate, acetyl trioctyl citrate or mixtures thereof.

15. Pharmaceutical composition according to claim 1, wherein the total amount of alkyl esters of citric acid is 0.1 to 10 times the amount of taxane by weight.

16. (canceled)

17. Pharmaceutical composition according to claim 1, wherein the amount of ethanol is between 5% by weight and 15% by weight.

18. (canceled)

19. Pharmaceutical composition according to claim 1, comprising at least one acid adjusting the pH of the composition to a value between 2 and 6.

20-21. (canceled)

22. Pharmaceutical composition according to claim 1, which is presented in a pre-filled syringe, vial, ampoule, bottle or bag for perfusion.

23. Pharmaceutical composition according to claim 1, wherein parenteral administration is performed by bolus, infusion and/or intravenous perfusion.

24. A method for the treatment of cancerous tumors which comprises the administration of a therapeutically effective amount of a pharmaceutical composition according to claim 1.

25. (canceled)

26. Composition for perfusion characterized in that it is a micro-emulsion or micro-suspension obtained by direct mixing of the composition according to claim 1 and an aqueous carrier suitable for intravenous perfusion.

27.-29. (canceled)
